(19) United States
(12) Patent Application Publication
(10) Pub. No.: US 2022/0160480 A1
(43) Pub. Date: May 26, 2022

(54) Dental Post

(71) Applicant: Marvis Interventional GmbH, Frechen (DE)

(72) Inventor: Klaus Düring, Frechen (DE)

(21) Appl. No.: 17/437,069

(22) PCT Filed: Mar. 6, 2020
(86) PCT No.: PCT/EP2020/056061

§ 371 Date: Sep. 8, 2021

(30) Foreign Application Priority Data
Mar. 8, 2019 (DE) 10 2019 106 034.6

(51) Int. Cl. A61C 13/30; A61K 6/844; A61K 6/17

(52) U.S. Cl. CPC A61C 13/30; A61C 2201/005; A61K 6/17; A61K 6/844

(57) ABSTRACT

The invention relates to a dental post formed from a composite of fibers and a matrix material, wherein X-ray markers are embedded in the matrix material and the X-ray markers are formed from visible and/or UV light reflective material. According to another aspect of the invention, the X-ray markers are concentrated in a central region of the dental post. The dental post has very good mechanical properties and allows visible and/or UV light to pass through to cure an adhesive disposed on the dental post.

The present invention relates to a dental post.

Dental posts can be used to rebuild teeth that have been treated with root canal treatment so that an artificial tooth replacement, e.g. a crown, can be placed stably on a remaining tooth. U.S. Pat. No. 5,890,904 describes a dental post comprising a core of fibers and a sheath consisting of fibers opaque to X-rays.

U.S. Pat. No. 6,012,924 discloses a dental post that is opaque to X-rays. The dental post comprises a core of fibers. The sheath consists of a matrix of resin, whereby the refractive index of the matrix is adjusted by doping with metal oxides. This allows to modulate the opacity to X-rays.

A dental post according to DE 600 25 501 T2 is characterized by a core of composite material of fibers embedded in a resin matrix, the fibers being opaque to X-rays and the difference in refractive indices between fibers and resin being less than 0.15. Only a minimal amount of fibers is used to impart the mechanical properties. In addition to metal oxides, quartz glass can also be used. This dental post is transparent to visible light and opaque to X-rays.

U.S. Pat. No. 8,039,101 B2 describes a radiopaque dental prosthesis made of a composite material formed of a fiber embedded in resin, the resin containing at least one radiopaque component.

The radiopaque component consists of nanoparticles with a diameter smaller than 60 nm that transmit part or all of the wavelengths in the range of 400 to 600 nm of an incident radiation. UV radiation is hardly affected. Numerous metals and metal oxides are proposed for the radiopaque component.

In FR 2,892,301 A1 the dental post is characterized by the matrix containing a photochromic pigment. This allows the matrix to be temporarily colored when the dental post is exposed to UV or visible radiation.

U.S. Pat. No. 9,814,655 B2 discloses a dental filling material having a core and a shell. The core consists of a core material and the shell consists of a shell material. The core material and the shell material are each a different material, with the core material having a larger particle size than the shell material.

DE 10 2015 220 373 A1 describes a curable dental material. The dental material comprises polymerizable monomers, particulate fillers and auxiliary materials. The fillers consist of particles of ytterbium fluoride and/or barium sulphate.

DE 10 2008 042 021 B3 describes an endodontic post with a one-piece base body for insertion into a curved root canal. This comprises a polymer matrix which is reinforced with fibers which are embedded in the polymer matrix, wherein the base body has zones of different bending and torsional stiffness and wherein at least one zone has a high bending and torsional stiffness and at least one zone has a reduced bending and torsional stiffness, wherein the fibers are embedded in the polymer matrix parallel to the longitudinal axis. The endodontic pin is characterized in that the polymer matrix reinforced with fibers contains different concentrations of reflection/absorption particles for reflection and absorption of laser beams over the length and cross-section of the base body, so that the different bending and torsional stiffness is thereby achieved and wherein a higher proportion of the reflection/absorption particles is contained in the at least one zone of high bending and torsional stiffness than in the at least one zone of reduced bending and torsional stiffness.

The object of the present invention is to create a dental post which can be produced simply and inexpensively, has good mechanical properties (such as strength and flexibility) and can be positioned in the tooth stump in a controlled and precise manner.

A further object of the present invention is to provide a dental post which is reliably and precisely detectable by means of an X-ray method.

A further object of the present invention is to create a dental post that meets high aesthetic requirements.

A further object of the present invention is to provide a dental post which can be reliably and quickly inserted and bonded by means of light-curing adhesives.

One or more of the objects are solved with a dental post according to one of the independent claims. Advantageous embodiments of the invention are indicated in the respective subclaims.

A dental post according to the invention is formed from a composite material comprising fibers and a matrix material. X-ray marker particles are embedded in the matrix material, and the X-ray marker particles are formed of visible or UV light reflective material, and wherein the X-ray marker particles are uniformly distributed in the composite material forming the dental post, and wherein the X-ray marker particles are adapted both to reflect light and to be visible in X-ray examinations.

Because the dental post has X-ray markers in the matrix material, it is not necessary to use fibers doped with X-ray markers. Fibers doped with X-ray markers are visible in an X-ray process but have poorer mechanical properties than corresponding undoped fibers. Therefore, the fiber properties can be fully adjusted to the mechanical properties, and thus the best possible mechanical properties for a dental post can be achieved.

Dental posts are short pins with a length of, for example, about 1 to 3 cm and a diameter of, for example, about 1 mm to 3 mm. The volume of such a dental post is limited. Therefore, it is important that the small amounts of fibers that can be provided in the dental post provide it with good mechanical properties, including high strength and flexibility. Since the X-ray markers are located in the matrix material, non-doped fibers can be used, which usually have better mechanical properties than doped fibers.

Since particles that reflect visible or UV light are used as X-ray markers, the visible or UV light is not absorbed by the X-ray markers.

This allows visible or UV light to be directed through the dental post to an adhesive located at the bottom and walls of the dental post to effect the curing of such a light-curing adhesive. This allows rapid bonding of such a dental post in the tooth stump without long curing times.

A curing light device does not necessarily illuminate the dental post evenly. The particles reflect and scatter the light in the dental post, creating an even distribution of light. Thus, an even illumination also of the surface of the dental post is achieved.

In the endodontic pin provided in DE 10 2008 042 021 B3 it is envisaged that a base body and/or the fiber-reinforced polymer matrix contains different concentrations of reflection/absorption particles for the reflection and absorption of laser beams over the length and cross-section of the base body, so that the different bending and torsional stiffness is thereby achieved and wherein a higher proportion of the reflection/absorption particles is contained in the at least one zone of high bending and torsional stiffness than in the at least one zone of reduced bending and torsional stiffness.

In contrast, in the dental post according to the invention, it may be provided that the composite material forming the dental post has an approximately constant concentration of X-ray marker particles over its entire length, so that the composite material forming the dental post has a homogeneous stiffness and/or homogeneous material properties over its entire length.

The particles are preferably uniformly distributed in the composite material forming the dental post. In the context of the present invention, the expression that the particles are preferably uniformly distributed is understood to mean that the particles embedded in the matrix material of the composite material may be approximately uniformly distributed along a longitudinal direction of the dental post forming material and/or that the particles embedded in the matrix material of the composite material may be approximately uniformly distributed transversely to the longitudinal direction of the dental post forming material. That is, a uniform distribution is present when the composite material forming the dental post is homogeneously doped with marker particles along the longitudinal axis.

The composite material forming the dental post is designed with a consistent or constant, resp., flexibility.

Furthermore, according to DE 10 2008 042 021 B3, in addition to the reflection/absorption particles for reflection and absorption of laser beams, nanodisperse materials and/or X-ray contrast agents are introduced into the endodontic pin as further fillers, preferably carbon nanotubes, hydroxyapatite, SiO₂, nanodisperse Ag, CeO, SnO₂ and/or O₂, WO₃.

The reflection/absorption particles are thus not used as X-ray markers, but only to modulate the mechanical properties. The aforementioned materials must therefore necessarily be additionally contained in this endodontic pin.

As shown above, on the other hand, it is provided according to the invention that means for X-ray visualization and for reflecting visible and/or UV light consist of the X-ray markers. This means that the X-ray marker particles simultaneously perform both the task of reflecting light and making it visible during X-ray examinations or are designed accordingly.

The particles can be formed from metals, such as tungsten or tantalum. Preferably, they are metals such as platinum, iridium or rhenium. In particular, the particles are preferably formed of gold. Alternatively, the particles may be formed of mixtures or alloys of these metals. The material from which the particles are made preferably has a high density and a high atomic number to ensure high opacity to X-rays. Furthermore, the material has a light-reflecting and preferably a shiny surface. The surface can be smooth and reflective. However, it may also be rough so that light is diffusely reflected. It is advantageous if the proportion of light absorbed at the surface of the particles is as small as possible, so that as large a proportion of the light as possible can be transmitted through the dental post. This applies equally to reflective and rough surfaces. Reflective surfaces of the particles lead to a shiny-white image in the X-ray image (with white representation of the X-ray marker) compared to a dull-white image of other X-ray markers such as ytterbium fluoride or barium sulphate). Therefore, specular X-ray markers are particularly preferred because they allow easier and better detectability in the X-ray image.

Ytterbium fluoride produces a dull-white image with good contrast in X-ray imaging. Gold, on the other hand, produces a shiny-white image with even better contrast in X-ray imaging, making it easier to identify and allowing the use of smaller amounts of X-ray marker. According to another aspect of the present invention, there is provided a dental post formed from a composite of fibers and a matrix material, wherein the X-ray markers have a higher concentration in a central region of the dental post than in a peripheral region of the dental post, or the X-ray markers are provided exclusively in the central region.

In the context of the present invention, a central region is understood to be a region which extends approximately over the entire length of the dental post and which is approximately cylindrical in shape.

The edge area also extends approximately over the entire length of the dental post, whereby this is designed concentrically surrounding the central area. I.e. the edge area is approximately tubular.

Such an arrangement in the central region is also referred to as a uniform distribution in the context of the present invention, since the dental post is homogeneously doped with X-ray marker particles in the central region along its longitudinal axis, and preferably also transversely thereto.

The placement of markers, which are usually formed of particles, in the matrix material of a composite material degrades the mechanical properties (such as strength and flexibility) of the composite material. Since the markers are mainly concentrated in the central area of the dental post, the weakening of the central area is stronger than that of the edge area of the dental post. The edge area has a stronger influence on the overall strength of the dental post due to its distance from the center. The concentration of X-ray markers in the central area of the dental post thus improves the overall mechanical properties, such as strength and flexibility, compared to a dental post in which the same amount of marker particles is evenly distributed in the matrix material. A dental post has to support a tooth stably in the jaw for decades and must not break. Therefore, the mechanical properties of a dental post are extremely important.

By providing a central doped area, a smaller amount of expensive X-ray marker particles, which can be made of gold or platinum, for example, is needed. In this way, less gold or platinum is used, while at the same time a good contrast is produced in an appropriate X-ray examination, resulting in lower costs for such a dental post.

A dental post made of a composite material of fibers and a matrix material, in particular a matrix material made of a resin or a polymer, has much more similar mechanical properties to dentin than conventional dental posts made of metal. With such a dental post it is easier to achieve a permanent fixation of a dental prosthesis in the jaw.

Preferably, the X-ray markers are located exclusively in the central area of the dental post. The central area of the dental post can extend from a center line of the dental post in the range of up to about 0.8 times, preferably 0.7 times, preferably 0.6 times, and in particular 0.5 times the radius of the dental post. The remaining area forms the edge area.

The fibers can be made of a material that is not doped with X-ray markers. Such fibers usually have better mechanical properties than fibers doped with X-ray markers.

The fibers can be glass fibers, carbon fibers, ceramic fibers, silica carbide fibers and/or basalt fibers.

The matrix material is preferably formed from a polymer material, in particular epoxy resin, chemically reactive polymerizing plastics or thermosets, e.g. polyester resins, vinyl ester resins, methacrylate resins, phenacrylate resins, spatially cross-linking polyurethanes and/or formaldehyde resins. The matrix material may be composed of one of these materials or a mixture of two or more of these materials.

The X-ray markers can be formed of particles with a size of at least 0.5 μm, preferably at least 1 μm, and especially at least 5 μm. The larger the particles, the more marker material can be embedded in the matrix material and the better the dental post is visible under X-ray radiation.

The X-ray markers may be formed of particles no larger than 500 μm or 250 μm or 100 μm or 50 μm or 20 μm or 10 μm or 5 μm or 2 μm and in particular no larger than 1 μm. The smaller the particles, the less resistance they offer to light that is to be passed through the dental post to cure an adhesive.

The X-ray markers may be provided at a concentration of at least 2%, preferably at least 5% or at least 10% or at least 15% and in particular at least 20% of the relative weight of the X-ray markers to the weight of the matrix material in which they are embedded. The more X-ray markers are provided in the dental post, the better the X-ray marker is visible under X-ray radiation.

The X-ray markers are preferably embedded in the matrix material at a concentration of no more than 200%, preferably no more than 100% or no more than 70% or no more than 60% and in particular no more than 50% of the relative weight of the X-ray markers to the weight of the matrix material. The lower the concentration of X-ray markers, the better light can be transmitted through the dental post for curing of the adhesive.

The X-ray markers can be made of a material with an atomic number of at least 70 and in particular at least 77. As a rule, the higher the atomic number, the better the X-ray markers are visible under X-ray radiation.

The X-ray markers can be formed from a material with a density of at least 15 g/cm³. The higher the density of the material, the better the X-ray markers are visible in an X-ray imaging process.

The X-ray markers can be formed from spherical particles. Spherical particles have the advantage that the surface area in terms of volume is small compared to other shapes. This allows the particles to contain a lot of marker material with a small surface area, so that they provide good visibility in an X-ray imaging procedure and yet offer little resistance to light transmitted through the dental post.

The X-ray markers can also be formed from elongated particles. In this case, however, it is useful if the X-ray markers are arranged in the longitudinal direction of the dental post so that light directed in the longitudinal direction of the dental post is impaired as little as possible and, in addition, the particles can be placed well between individual fibers of the dental post.

The X-ray markers can be made of particles with a shiny surface. This ensures that light is mainly reflected and not absorbed at the surface of the X-ray markers.

In this way, a dual mode of operation is achieved, since the composite material forming the edge region is designed to be transparent to light and the composite material forming the central region absorbs the X-rays.

Such an arrangement in the peripheral region or in the edge region is also referred to as a uniform distribution in the context of the present invention, since the dental post can be homogeneously doped with marker particles in the edge region along its longitudinal axis, and preferably also transversely thereto.

The edge area can thus be undoped or have a lower concentration than the central area.

The matrix material of the edge area can be translucent or slightly milky or more milky, whereby the light transmission of the matrix material must be guaranteed to a sufficient degree. Preferably, only the outermost layer or only the surface of the matrix material of the edge region is slightly milky or more milky. The advantage of such a superficial or homogeneous milky design of the matrix material of the marginal area is that the color of the X-ray marker particles arranged in the central area of the dental post does not show through to the outside or only to a lesser extent. Aesthetically preferred embodiments are thus achieved. The superficial or homogeneous milky finish of the matrix material of the marginal area can be achieved by suitable materials and processing procedures, e.g. by a corresponding cross-linking of resins or polymers or also by a surface treatment of the matrix material of the edge area.

The invention is explained in more detail below by means of the examples shown in the drawings. The drawings show schematically:

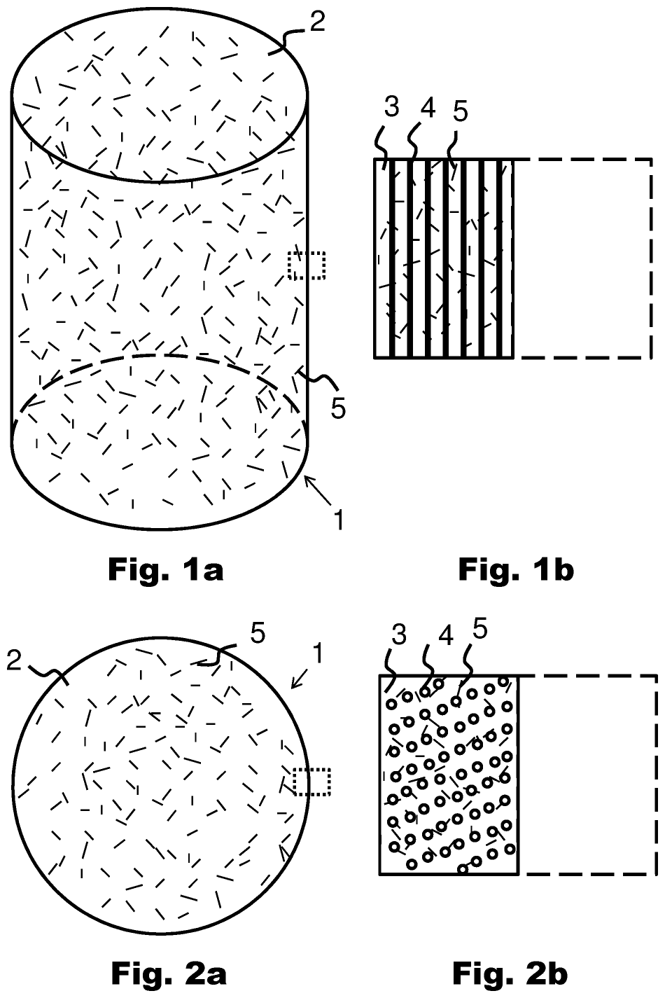

FIG. 1a a section of a dental post in isometric view according to a first embodiment,

FIG. 1b a section of an edge region in side view of a dental post according to a first embodiment,

FIG. 2a a cross-section of a dental post in a top view according to a first embodiment,

FIG. 2b section of an edge region of a dental post in a top view according to a first embodiment,

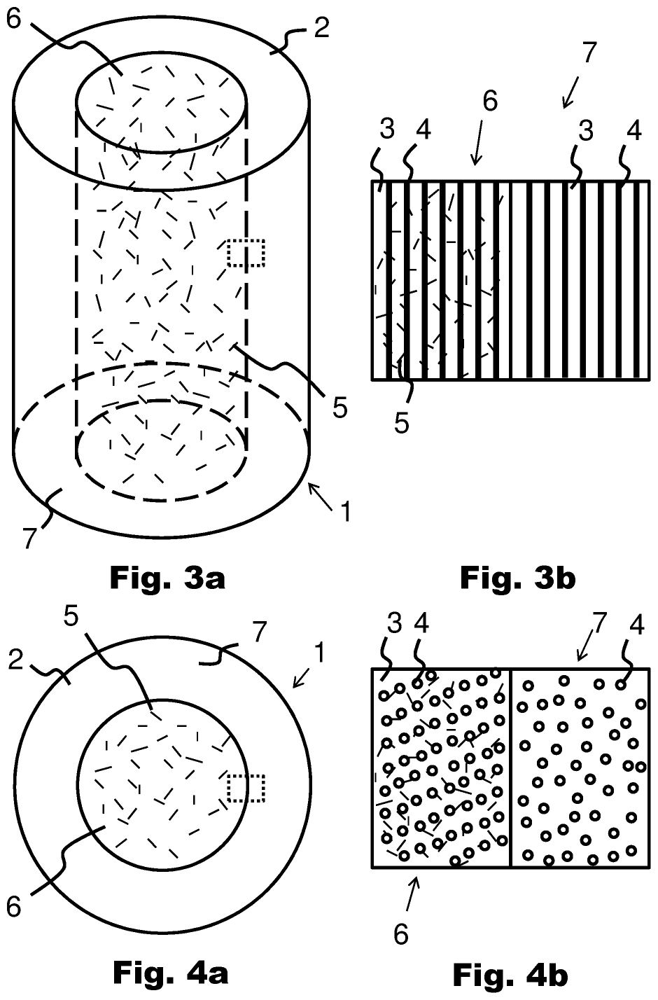

FIG. 3a section of a dental post in isometric view according to a second embodiment,

FIG. 3b section of an edge region in side view of a dental post according to a second embodiment,

FIG. 4a cross-section of a dental post in a top view according to a second embodiment, and

FIG. 4b section of an edge region of a dental post in a top view according to a second embodiment.

A first example of an embodiment (FIGS. 1a-2b) is explained below.

A dental post 1 according to the invention is designed as a rod-shaped body. The diameter of the dental post 1 is at least 0.8 mm, preferably at least 1.0 mm and in particular at least 1.2 mm. The thinner the dental post is, the easier it can be inserted into a corresponding borehole. The diameter of the dental post 1 is at most 3.5 mm, preferably at most 3.0 mm and in particular at most 2.5 mm. The thicker the dental post is, the more stable it is and can hold a dental prosthesis, e.g. a crown, stably in the long term.

The dental post 1 is at least 5 mm, preferably at least 7 mm, in particular at least 10 mm long. The dental post 1 is at most 30 mm, preferably at most 25 mm, in particular at most 22 mm long.

The dental post 1 is made of a composite material 2. The composite material 2 has a matrix material 3 and fibers 4. The matrix material 3 is preferably a polymer, in particular epoxy resin. However, other chemically reactive polymerizing plastics can also be used, such as polyester resins, vinyl ester resins, methacrylate resins, phenacrylate resins, spatially cross-linking polyurethanes or formaldehyde resins. The matrix material is nearly transparent to visible and/or UV light but may have a slightly milky finish for visual coverage of a colored X-ray marker in the central region. The matrix material may be composed of one of these materials or a mixture of two or more of these materials.

The fibers 4 run approximately parallel to the rod direction of the dental post 1. They are arranged approximately evenly. The fibers preferably extend over the entire length of the dental post.

The fibers 4 can, for example, be formed from glass fibers, ceramic fibers, silica carbide fibers, carbon fibers and/or basalt fibers.

The fibers can consist of a single material or be composed of a mixture of two or more of these materials. In principle, stiff fibers, especially glass fibers, are preferred. However, it may also be appropriate to combine fibers of one of the above-mentioned stiff fiber types with a softer but tensile fiber, such as an aramid fiber. The stiff fibers are preferably arranged radially on the outside and the softer fibers radially on the inside.

If the fibers are 4 carbon fibers, then restrictions regarding the aesthetic properties may have to be accepted at higher concentrations, as they may darken the dental post and the black color may show through to the surface of the dental prothesis. To minimize this coloration, a slightly milky or more milky surface finish can again be used. Other suitable and less stiff fibers are made of polyethylene terephthalate (PIT), for example Dacron®, chromatic polyamides, for example Kevlar® or vegetable fibers, for example silk, sisal, hemp, which however may be less stable. These fibers are preferably combined with stiff fibers as explained above. Preferably, the material of the fibers is not doped.

Several fibers are combined in so-called rovings and are impregnated as such with the matrix material during production. A dental post usually comprises 3 to 10 rovings.

These rovings can have a weight per length of at least 500 Tex (1 Tex=1 gram/1,000 metres) or at least 750 Tex or at least 1,000 Tex. Preferably, the weight per length is not greater than 1,500 Tex or 2,000 Tex and in particular not greater than 2,500 Tex.

Particles 5 are embedded in the matrix material 3. The particles 5 are formed as X-ray markers. The particles 5 are preferably evenly distributed in the dental post 1 but could also be unevenly distributed. The particles 5 are formed of metals, such as tungsten or tantalum. Preferably, they are metals such as platinum, iridium or rhenium. In particular, the particles 5 are preferably formed of gold. Alternatively, the particles 5 may be formed of mixtures or alloys. The material of which the particles 5 are made preferably has a high density, preferably greater than 15 grams per cm³, and/or a high atomic number, preferably greater than 70, in order to ensure a high opacity to X-rays.

The composite material forming the dental post 1 has an approximately constant concentration of X-ray marker particles over its entire length, so that the composite material forming the dental post has homogeneous stiffness and/or homogeneous material properties over its entire length.

The particles are preferably uniformly distributed in the composite material forming the dental post 1. The particles embedded in the matrix material of the composite material are distributed approximately uniformly along a longitudinal direction of the dental post forming material. The particles embedded in the matrix material of the composite material are also approximately uniformly distributed transversely to the longitudinal direction of the material forming the dental post. That is, a uniform distribution is present when the composite material forming the dental post is homogeneously doped with marker particles along the longitudinal axis.

The particles are preferably round or spherical or ellipsoidal but can also be elongated, rod-shaped or flat. Approximately round shapes are advantageous in the production process because they are most evenly distributed in the matrix material and can most easily pass through the openings of production tools. Approximately spherical particles have the largest volume with a minimum surface area. This allows a lot of marker material to be introduced into the matrix material with approximately spherical particles, while keeping the disturbance of light penetration low due to the small surface area.

Elongated particles, on the other hand, have the advantage that they can be arranged between fibers approximately parallel to them and thus fit between closely adjacent fibers without greatly interfering with the arrangement of the fibers. In addition, the cross-sectional area in the longitudinal direction is small when the elongated particles are aligned in the longitudinal direction, so that there is little obstruction to the passage of light in the longitudinal direction.

Elongated particles are therefore very advantageous if they are aligned in the longitudinal direction. Otherwise, spherical particles are preferred, as these do not have to be aligned when they are introduced into the matrix material and still have little effect on the passage of light and a relatively large amount of marker material can be introduced into the matrix material.

Particles 5 of gold, which has an atomic number of 79, have a density of about 19.3 g/cm³ and have a shiny golden surface.

Particles 5 made of platinum, which has an atomic number of 78, have a density of about 21.5 g/cm³ and have a shiny silver surface. Particles 5 of iridium, which has an atomic number of 77, have a density of about 22.6 g/cm³ and have a shiny silver-white surface. Particles 5 of rhenium, which has an atomic number of 75, have a density of about 21.0 g/cm³ and have a shiny silver surface. Particles 5 of tantalum, which has an atomic number of 73, have a density of about 16.7 g/cm³ and have a graphite-grey shiny surface. Particle 5 of tungsten, which has an atomic number of 74, has a density of about 19.3 g/cm³ and has a shiny grey-white surface.

The higher the atomic number and the greater the density of the material of the particles, the better these particles are detected under X-rays. Therefore, particles 5 made of a material with an atomic number of at least 70 and in particular of at least 77 are preferred. A material with a density of at least 15 g/cm³ is preferred.

Furthermore, the particles 5 have a light-reflecting and preferably shiny surface. The surface of the particles 5 reflects light with a wavelength of at least 100 nm, preferably of at least 300 nm, and in particular of at least 400 nm. The surface of the particles 5 reflects light with a wavelength of at most 900 nm, preferably of at most 800 nm, and in particular of at most 750 nm.

The surface of the particles 5 can be structured three-dimensionally in such a way that light is reflected diffusely.

The particles 5 have a size of at least 0.5 μm, preferably at least 1 μm, and in particular at least 5 μm. The particles 5 preferably have a size which is not larger than 500 μm or 250 μm or 100 μm or 50 μm or 20 μm or 10 μm or 5 μm or 2 μm and in particular not larger than 1 μm. If the particles 5 were smaller than the limits specified here, they would no longer be able to reflect visible or UV light, as the wavelength would be larger than the particles 5. If the particles 5 were larger than the limits specified here, they would reflect the light on one side.

The smaller the particles 5 are, the less they block light passing through the dental post. The larger the particles are, the more marker material can be introduced into the matrix material and the better the dental post is visible under X-ray radiation. These are therefore opposing requirements that have to be balanced depending on the materials used and the size of the dental post.

The concentration of the particles 5 is given below as the relative weight of the particles to the weight of the matrix material 3 in which they are embedded. If, for example, the dental post contains areas of matrix material, as in the embodiment example explained below, in which no particles 5 are provided, then this matrix material is not taken into account when determining the concentration. The concentration of the particles 5 is at least 2%, preferably at least 5 or 10% or 15%, and in particular at least 20%. The concentration of the particles 5 is at most 200%, preferably at most 100% or 70% or 60% and in particular at most 50%. The higher the concentration, the higher the absorption of X-rays. However, this makes the matrix material 3 more brittle.

The particles 5 are randomly oriented. Furthermore, the particles 5 in the respective area are preferably randomly distributed and arranged. The particles 5 can also be evenly or unevenly distributed or orderly aligned.

A second embodiment (FIGS. 3a-4b) is explained below, whereby the same elements as in the first embodiment are marked with the same reference signs. The above explanations apply to identical elements, unless otherwise stated below.

In the second embodiment again a dental post 1 is provided, which is formed from a composite material 2. The outer shape of the dental post 1 and the composite material 2 of the second embodiment are the same as those of the first embodiment.

In contrast to the first embodiment, the dental post 1 in this embodiment has a central region 6 and an edge region 7. The central region 6 has a higher concentration of particles 5 than the edge region 6. The central region 6 extends from a center line of the dental post 1 in a range up to at least about 0.05 times, preferably at least 0.1 times, or 0.2 times, and in particular at least 0.3 times the radius of the dental post 1. The central region 6 extends at most in a range of 0.8 times, preferably 0.7 times, or 0.6 times, and in particular 0.5 times the radius of the dental post 1. The respective remaining region forms the edge region 7.

The concentration of particles in the central region 6 is at least 2%, preferably at least 5%, or 10%, or 15%, and in particular at least 20%.

In the central area 6, the concentration of the particles 5 is at most 200%, preferably at most 100% or 70% or 60%, and in particular at most 50%. In the edge region 7, the concentration of the particles 5 is at least 0%, preferably at least 2%, or 3%, and in particular at least 5%. The concentration of the particles 5 in the edge region 7 is at most 100%, preferably at most 75%, or 50%, and in particular at most 30%.

The central area 6 extends approximately over the entire length of the dental post 1 in the longitudinal direction. This area 6 is approximately cylindrical.

The edge area 7 also extends approximately over the entire length of the dental post in the longitudinal direction, whereby this is formed concentrically surrounding the central area. I.e. the edge area 7 is approximately tubular.

Such an arrangement in the central region 6 is also referred to as a uniform distribution in the context of the present invention, since the dental post 1 is homogeneously doped with X-ray marker particles in the central region 6 along its longitudinal axis, and preferably also transversely thereto.

An arrangement of X-ray marker particles in the peripheral region or in the edge region 7 also represents as a uniform distribution, since the dental post 1 can be homogeneously doped with marker particles in the edge region 7 along its longitudinal axis, and preferably also transversely thereto.

The edge area 7 can thus be undoped or have a lower concentration than the central area 6.

Another possibility of the second embodiment is that the fibers in the central area 6 have a lower density than in the edge area 7. As already described above, the edge area has a stronger influence on the overall strength of the dental post due to its distance from the center. Thus, the fiber density in the interior may be lower. This is advantageous in that an increased concentration of particles 5 can be used without reducing the concentration of the matrix material 3.

The procedure for distributing light in the dental post 1 is described below. If a dental post 1 is to be cemented into the tooth stump, the dental post 1 is illuminated with a conventional curing light device. This allows the adhesive around the dental post to be polymerized by visible or UV light.

The light of the curing light device penetrates through the transparent or slightly milky or more milky matrix material 3, whereby a substantial part penetrates through the cross-sectional area at the upper end of the dental post into the matrix material 3. With only a superficial milky design of the matrix material 3 in the edge area, the highest penetration rate for the incident light is thus achieved, which is only exceeded by a completely translucent design of the matrix material in the edge area 7. Part of the light then hits the particles 5. The particles 5 reflect the light in random directions. The reflected light then partly hits particles 5 again, which reflect the light again. This process repeats until the light has either been absorbed or exits the dental post 1. Due to the multiple reflection of the light, the light is diffusely guided through the dental post 1.

### LIST OF REFERENCE SIGNS

1 Dental post

2 Composite material

3 Matrix material

4 Fibers

5 Particles

6 Central area

7 Edge area

1. A dental post formed of a composite material of fibers and a matrix material, wherein X-ray marker particles are embedded in the matrix material and the X-ray marker particles are formed of visible and/or UV light reflecting material, and wherein the X-ray marker particles are uniformly distributed in the composite material forming the dental post, and wherein the X-ray marker particles are provided both to reflect light and for visualization in X-ray exons.

2. The dental post according to claim 1, wherein
the X-ray marker particles are made of gold, platinum, iridium, rhenium, tungsten or tantalum, or a mixture or alloy of two or more of gold, platinum, iridium, rhenium, tungsten or tantalum.

3. The dental post according to claim 1,
wherein the X-ray marker particles have a higher concentration in a central region of the dental post than in an edge region of the dental post.

4. The dental post according to claim 3,
wherein
the X-ray marker particles are arranged exclusively in the central region of the dental post.

5. The dental post according to claim 3,
wherein
the central region extends from a center line of the dental post into a region up to approximately 0.8 times the radius of the dental post, whereas the remaining region forms the edge region, so that the central region is approximately cylindrical and the edge region is approximately tubular.

6. The dental post according to claim 1,
wherein
the matrix material of the edge region is translucent or milky,
wherein the matrix material in this region has a certain translucency, or
wherein preferably a slightly milky or more milky formation only of an outermost layer, or only of the surface of the matrix material of the edge region is provided, so that the color of the X-ray marker particles arranged in the central region of the dental post does not show through to the outside or only to a lesser extent.

7. The dental post according to claim 1,
wherein
the fibers are made of a material not doped with X-ray markers.

8. The dental post according to claim 1,
wherein
the fibers are glass fibers, carbon fibers, ceramic fibers, silica fibers and/or basalt fibers.

9. The dental post according to claim 1,
wherein
the fibers run in the longitudinal direction of the dental post and/or extend over the entire length of the dental post.

10. The dental post according to claim 1,
wherein
the matrix material is formed
from a polymer material, in particular epoxy resin and/or a chemically reactive polymerizing plastic or thermoset, such as polyester resin, vinyl ester resin, methacrylate resin, phenacrylate resin, spatially cross-linking polyurethane and/or formaldehyde resin.

11. The dental post according to claim 1,
wherein
the X-ray marker particles have a size of at least 0.5 μm.

12. The dental post according to claim 1,
wherein
the X-ray marker particles have a size of not more than 500 μm.

13. The dental post according to claim 1,
wherein
the X-ray marker particles are provided with a concentration of at least 2% of the relative weight of the X-ray marker particles to the weight of the matrix material in which they are embedded.

14. The dental post according to claim 1,
wherein
the X-ray marker particles are provided with a concentration of at most 200% of the relative weight of the X-ray marker particles to the weight of the matrix material in which they are embedded.

15. The dental post according to claim 1,
wherein
the X-ray marker is formed from a material with an atomic number of at least 70 and/or from a material with a density of at least 15 g/cm³.

16. The dental post according to claim 1,
wherein
the X-ray marker particles are approximately spherical particles.

17. The dental post according to claim 1,
wherein
the X-ray marker particles are elongated particles which are predominantly arranged approximately longitudinally in the dental post.

18. The dental post according to claim 1,
wherein
the X-ray marker particles are particles with a shiny surface.
